(19) United States
(12) Patent Application Publication
(10) Pub. No.: US 2021/0215577 A1
(43) Pub. Date: Jul. 15, 2021

(54) Portable System and Process Thereof to Rapidly Detect the Presence, Family of Origin and Ratio of Any Bacteria and Estimate the Endotoxin-Producing Bacteria Levels of a Water Sample

(71) Applicant: Nephros Inc., South Orange, NJ (US)

(72) Inventors: Daron Evans, Woodside, CA (US); Kimothy Smith, Incline Village, NV (US)

(21) Appl. No.: 17/150,117

(22) Filed: Jan. 15, 2021

Related U.S. Application Data
(60) Provisional application No. 62/961,513, filed on Jan. 15, 2020.

(51) Int. Cl. G01N 1/14; G01N 1/40; C12Q 1/689; B01D 63/02; B01D 69/02; B01D 69/08

(52) U.S. Cl. CPC G01N 1/14; G01N 1/4077; C12Q 1/689; B01D 63/02; B01D 69/02; B01D 2325/02; G01N 2001/4088; B01D 2313/243; B01D 2313/16; B01D 2313/44; B01D 2319/04; B01D 69/08

(57) ABSTRACT

Disclosed herein is a high efficiency portable filtration device and a method for rapidly filtering, concentrating and detecting the presence of any waterborne pathogens, such as bacteria, in a liquid (water) sample, measure the total numbers of bacteria present, and to estimate the endotoxin-producing bacteria levels in the water sample.

### CROSS REFERENCE TO RELATED APPLICATION

This application claims the benefit of U.S. Provisional Application No. 62/961,513, filed Jan. 15, 2021, the entire teachings of which are incorporated herein by reference as is presented in its respective entirety.

### TECHNICAL FIELD

The present invention is directed to a system and process for detecting the presence, genus and ratio of Gram-negative bacteria in a water sample, particularly, to a portable apparatus and process for rapidly filtering, concentrating and detecting any bacteria in a water sample, more particularly, to a high efficiency filtration system and a method thereof for analysis of a water sample to detect the presence of all bacteria in the water sample, measure the total numbers of bacteria present, and to estimate the endotoxin-producing bacteria levels in the water sample. Also disclosed herein is a kit to detect the presence of bacteria in a water sample, comprising the disclosed filtration device and a molecular detection device. Also disclosed herein is a hardware mobile electronic device and a software application that analyze a water sample to detect the presence, family of origin and ratio of bacteria, and wirelessly transmits such information to users. Also disclosed herein is a methodology to utilize data regarding the presence, family of origin and ratio of any bacteria in a water sample to provide an estimate of endotoxin-producing bacteria levels in a water sample.

### BACKGROUND

Many waterborne pathogens cause infections and human disease via ingestion of contaminated water. Various human parasites and pathogens are transmitted in this way, including protozoa, virus and bacteria. These parasites and pathogens can be transmitted via human fecal contamination of water used for drinking, bathing, recreation, harvesting of shellfish, or washing/preparation of foods. Warm stationary domestic water found in air conditioner cooling towers, inadequately chlorinated swimming pools and spas, hot water heaters, respiratory therapy equipment and shower heads, have been identified as sources of infectious outbreaks. The need for and adequacy of water purification and the safety of natural waters is paramount, and sources of such water reservoir are routinely monitored by standard microbiological tests for infectious flora.

Detection and analysis of waterborne pathogens can be employed for effective prevention of infectious outbreaks. The identity of an infectious species in a sample can be ascertained by comparing the nucleic acid present in the sample to the known nucleic acid sequence. Before making this comparison, however, the nucleic acids must be extracted from the sample, amplified, and then detected. Typically, these steps take place over the course of hours, or days in a laboratory. For example, amplification usually involves the polymerase chain reaction (PCR) as described in U.S. Pat. Nos. 4,683,202 and 4,683,195, herein incorporated by references as is presented in their entireties. To prepare for the nucleic acids amplification using conventional PCR, the biological sample containing nucleic acids must be treated with lyse solution and incubated.

Traditional methods and devices for extraction, amplification, and detection of nucleic acids are not typically designed to be performed in a mobile or field setting outside a specialized lab infrastructure. Extraction and amplification alone takes hours if not days, depending on the type of organism, the length of the nucleic acid strand, and the number of cycles. Absent tightly controlled test setting, contaminants can interfere with the nucleic acid polymerase enzymes used in replication, reducing the efficiency and fidelity of the amplification process. Therefore, there is an unmet need for a rapid, accurate and portable device and for detecting, quantifying and identifying target nucleic acids.

Thus, the current state of the art requires samples to be collected from remote locations and delivered to a centralized laboratory facility for detection of waterborne pathogens. This process typically takes up to two weeks to complete and cannot be implemented in a rapid, portable procedure on-site at the remote location.

The dialysis and pharmaceutical manufacturing industries commonly test for bacterial endotoxin. The limulus amebocyte lysate (LAL) test, used for bacterial endotoxin testing, is an in vitro assay used to detect the presence and concentration of bacterial endotoxins. Endotoxins, which are a type of pyrogen, are lipopolysaccharides present in the cell walls of gram-negative bacteria. The same sample collection and processing process for the LAL test requires fluid samples to be collected at various locations of interest, mailed overnight to a laboratory, and processes at a centralized laboratory. Preliminary results are released in 24 hours after receipt of a sample, and final results are released six to 7 days after receipt of the sample.

### SUMMARY

In one aspect, a method for rapidly isolating and detecting the presence and quantity of Gram-negative bacteria in a water sample, and, estimating the level of endotoxin-producing bacteria in the water sample, is provided and comprises the steps of:
filtering the liquid sample using a portable filtration device so that the bacteria is collected in a concentrated filtrate that comprises the bacteria and is contained within the portable filtration device;
introducing a lysing agent into the portable filtration device resulting in lysing of the waterborne pathogens and formation of a lysed bacterial solution that is contained within the portable filtration device;
removing an amount of the lysed bacterial solution from the portable filtration device;
introducing at least a portion of the removed amount of lysed bacterial solution into a molecular detection and amplification device that is configured to detect and quantity the presence and quantity of Gram-negative bacteria in a water sample; and,
estimating the endotoxin-producing bacteria level of the water sample from the analysis of the Gram-negative bacteria quantity present.

In another aspect, a portable kit is provided for rapidly isolating and detecting the presence of any Gram-negative bacteria. The kit includes a portable filtration device including a first port for receiving a liquid sample to be tested. The portable filtration device also includes a second port for venting air and a third port for discharging purified liquid. The portable filtration device further includes a plurality of hollow semi-permeable fibers for filtering the liquid sample and generating a filtrate within lumens of the hollow semi-permeable fibers. The filtrate contains the waterborne pathogens in a concentrated form. The kit also includes a syringe configured for being sealingly mated to the first port and configured to deliver a lysing agent into lumens of the plurality of hollow semi-permeable fibers for lysing of the waterborne pathogens and formation of a lysed waterborne pathogen solution. The syringe is further configured for removing the lysed waterborne pathogen solution from within the lumens. A molecular detection device is provided for analyzing the lysed waterborne pathogen solution and detecting whether target waterborne pathogens are present in the liquid sample.

### BRIEF DESCRIPTION OF THE DRAWING FIGURES

FIG. 1 is a schematic illustrating a portable system and process thereof to rapidly filter, concentrate, detect, amplify and characterize bacteria;

FIG. 2 is a schematic illustrating a portable system and process thereof to rapidly filter, concentrate, detect, amplify and characterize bacteria;

FIG. 3 is a cross-sectional view of a portable filtration device for use in the system;

FIG. 4 is a schematic of a portable filtration device according to another embodiment;

FIG. 5 is a schematic of a portable filtration device according to another embodiment;

FIG. 6 is a schematic of a portable filtration device and a device for delivering liquid to the portable filtration device;

FIG. 7 is a perspective view of a kit (contained in a hard shell case) including the system of the present disclosure;

FIG. 8 is a perspective view of a sealed package including components of the system;

FIG. 9 is a perspective view of another kit including the system of the present disclosure;

FIG. 10 is a graph of Cq versus CFU/ml and EU/ml of endotoxin of serial dilutions of E. coli in water analyzed using the qPCR assay for Gram-negative bacteria; and

FIGS. 11A-E are graphs of the comparison of the qPCR for the Gram-negative bacteria and another test for EU/ml. FIG. 11A-B is a comparison of the EU/ml using the two methods and FIG. 11C-E the EU/ml results using the approaches described herein and the alternative assay approach.

### DETAILED DESCRIPTION OF CERTAIN EMBODIMENTS

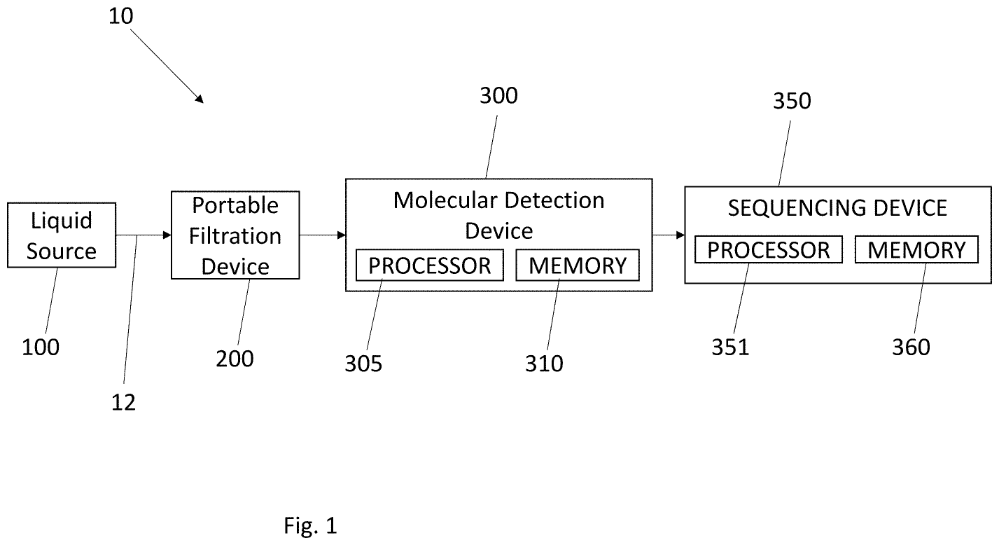

As described herein, the present disclosure is generally and broadly directed to a rapid, portable system and process to filter, concentrate, detect, amplify and characterize bacteria in liquids (liquid samples) using molecular biological methods, such as molecular detection devices, etc. For example, FIG. 1 is a schematic illustrating exemplary details of the present invention and more particularly, FIG. 1 illustrates a portable system 10 that is configured to rapidly filter, concentrate, detect, amplify and characterize bacteria in a liquid sample, in a culture-independent manner, and to estimate endotoxin levels in the liquid sample. The system 10 includes a number of components that are portable in nature and can be contained and packaged as a kit, as described herein. For example, the portable system 10 is configured to collect a liquid sample from a liquid source 100 and deliver it to a portable filtration device 200 that is configured to rapidly filter and concentrate the target pathogens and then the concentrated filtrate is removed from the filtration device 200 and delivered to a portable molecular detection device 300 for analyzing and amplifying bacterial genetic sequences in a liquid sample. By way of further example one or more primers and probes specific to Gram negative bacteria are used in quantitative PCR (qPCR) to estimate the amount of endotoxin. For instance, the amount of endotoxin the sample is calculated directly from the CFU calculated from the Cq of the amplification. The details and operation of the portable system 10, as well as all of the components thereof, are set forth below.

### Definitions

Unless defined otherwise, all technical and scientific terms used herein have the same meaning as commonly understood by those of ordinary skill in the art to which this disclosure belongs. Although any methods and materials similar or equivalent to those described herein can be used in the practice or testing of the present disclosure, the preferred methods and materials are described. For purposes of the present disclosure, the following terms are defined below.

The terms “bacteria” grammatical equivalents herein are meant any biomolecule or compound to be detected. Suitable biomolecules include, but are not limited to, proteins (including enzymes, immunoglobulins and glycoproteins), nucleic acids, lipids, lectins, carbohydrates, hormones, whole cells (including procaryotic and eucaryotic cells,), viruses, spores, etc.

The term “sample” in the present specification and claims are used in their broadest sense. On the one hand it is meant to include a specimen or culture. On the other hand, it is meant to include both biological and environmental samples. In addition, a “sample” may or may not contain nucleic acid.

The term “nucleic acid” or grammatical equivalents herein means at least two nucleotides covalently linked together. A nucleic acid of the present disclosure will generally contain phosphodiester bonds, although in some cases, as outlined below, nucleic acid analogs are included that may have alternate backbones, comprising, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977). Other analog nucleic acids include those with bicyclic structures including locked nucleic acids, Koshkin et al., J. Am. Chem. Soc. 120:13252-3 (1998); positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook.

The nucleic acids may be single stranded or double stranded, as specified, or contain portions, of both double stranded or single stranded sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, etc.

The term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH).

The term “probe” refers to an oligonucleotide (i.e., a sequence of nucleotides), whether occurring naturally as in a purified restriction digest or produced synthetically, which is capable of hybridizing to another oligonucleotide of interest. Probes are useful in the detection, identification and isolation of particular gene sequences.

As used herein, the term “portable” refers to a system or device or mobile device that can be easily carried or conveyed by hand by a person. As used herein, the term “mobile device” refers to a small portable device, typically having a display screen with touch input and/or a miniature keyboard, including for example a smart phone, tablet, laptop or other portable medical device.

The terms “application” or “software” are used herein in a generic sense to refer to any type of computer code or set of computer-executable instructions that can be employed to program a computer or other processor to implement various aspects of the present disclosure as discussed above.

Bacteria

As described herein, the portable system 10 is configured to collect, amplify and characterize any number of different bacteria (waterborne pathogens) that may be found in a liquid sample (i.e., “bacteria”). The system described herein does not amplify a specific set of target bacteria, rather it targets and amplifies a genetic segment that is believed to be generally ubiquitous in all bacterial. Differences within the ubiquitous genetic segment exist that, when sequenced and mapped versus a known database of bacterial genetic sequences, enable familial characterization of the bacteria in the liquid sample. As mentioned earlier, the portable system 10 utilizes a culture-independent DNA analysis method for detecting the presence of bacteria in the water sample, measuring the total numbers of bacteria present, and estimating the endotoxin-producing bacteria levels in the water sample.

Gram-negative bacteria are bacteria that do not retain the crystal violet stain used in the gram-staining method of bacterial differentiation. They are characterized by their cell envelopes, which are composed of a thin peptidoglycan cell wall sandwiched between an inner cytoplasmic cell membrane and a bacterial outer membrane.

Gram-negative bacteria can include, but are not limited to, Legionella, Burkholderia cepacian, Klebsiella, Pseudomonas spp, P. aeruginosa, Ralstonia picketti, Sphingomonas, Escherichia coli, Pseudomonas aeruginosa, Chlamydia trachomatis, and Yersinia pestis.

High Efficiency Filtration System and Method Thereof

As discussed herein, in one embodiment, the present disclosure is directed to the portable system 10 including the portable fluid filtration device 200 for use with device 300 for rapid analysis of one or more biological samples.

Now referring to FIG. 1, the liquid source 100 comprises any number of different types of sources of fluid that is to be analyzed using the portable system 10 for detection of bacteria. The liquid source 100 can take any number of different sizes and can be located at a variety of different locations. In general, the liquid source 100 can comprise any source of contaminated water. As previously discussed, contaminated water can be found in a wide array of locations including a source of drinking water, bathing water, recreation water (e.g., swimming pools and spas), air conditioning cooling towers, hot water heaters, respiratory therapy equipment, etc. In addition, natural water sources, such as springs, ponds, lakes, etc. can also become a source of contaminated water. In the dialysis or pharmaceutical manufacturing setting, the source could be ultrapure water and the level of contamination could be very low.

FIG. 1 illustrates that a first conduit 12 is used to deliver the liquid (water) from the liquid source 100 to the portable filtration device 200. In some settings, the first conduit 12 can be directly inserted into the liquid source 100, such as a water reservoir, cooling tower, pond, etc., or a sample can be taken and placed into a collection container and in that case, the first conduit 12 can be inserted into the collection container that holds the liquid sample. The first conduit 12 can be in the form of a flexible tube or the like. It will also be understood that the first conduit 12 can actually be formed of two or more tube segments that are coupled to one another with a connector or the like.

To deliver the fluid (liquid) from the liquid source 100 to the portable filtration device 200, a pump 210 can be used and is generally disposed along the first conduit 12 to controllably pump the fluid from the source 100 to the portable filtration device 200.

Any number of different types of pumps 210 can be used including automated pumps and manual pumps, such as hand pumps, etc.

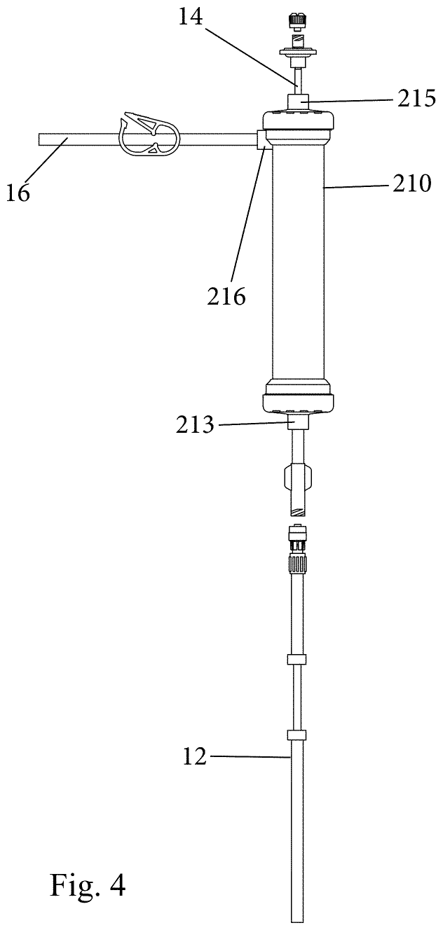

One exemplary type of pump 210 is a peristaltic pump. As is known, a peristaltic pump is a type of positive displacement pump used for pumping a fluid and also can be commonly referred to as a roller pump. The fluid is contained within a flexible tube fitted inside a circular pump casing. A rotor with a number of rollers attached to the external circumference of rotor compresses the flexible tube. As the rotor turns, the part of the tube under compression is pinched closed (occludes) thus forcing the fluid to be pumped to move through the tube. As the tube opens back to its natural state after the passing of the cam, fluid flow is induced to the pump. When a peristaltic pump 210 is used, the first conduit 12 can include a peristaltic pump tubing segment (FIG. 4) that is contacted by the rotor. The peristaltic pump 210 can be provided with a small footprint and can include a housing with an inlet and outlet connector for connecting to first conduit segments and can be powered by an electric power source or a battery power source. The pump 210 has conventional controls, such as on/off, speed, etc.

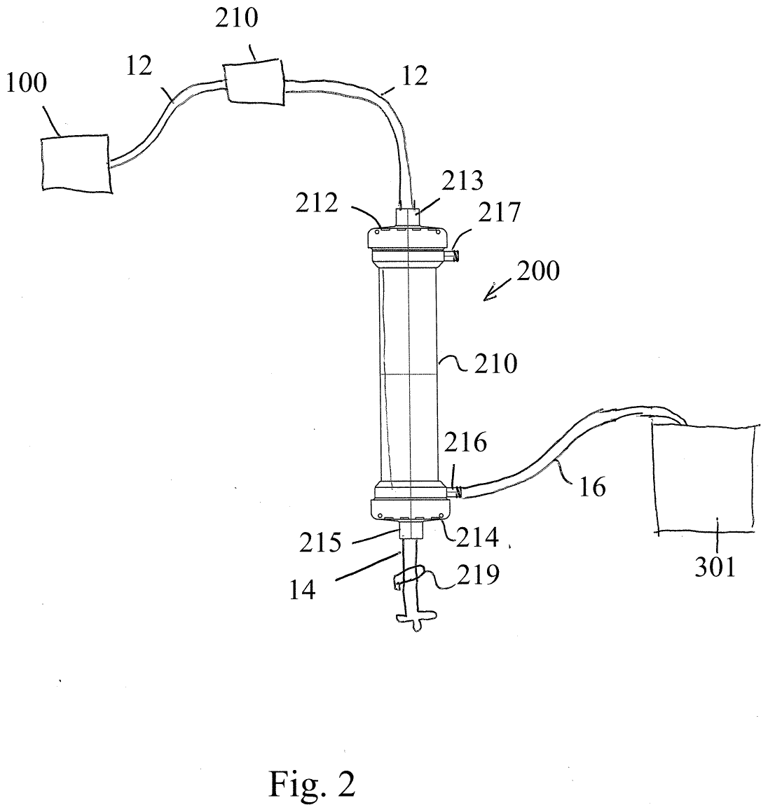

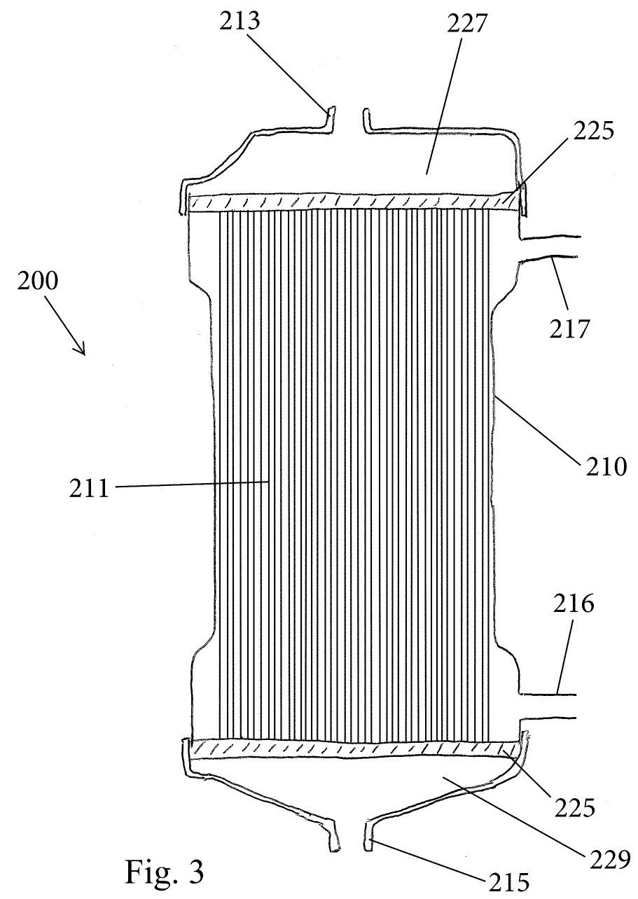

As shown in FIGS. 2 and 3, the portable filtration device 200 can come in any number of different forms that are suitable for the intended application. For example, the filtration device 200 is in the form of a cartridge that is defined by a housing 210. Housing 210 is preferably cylindrical in shape and is formed of a rigid plastic material. Housing 210 contains a longitudinal bundle of semi-permeable hollow fibers 211, as are known in the art. The semi-permeable hollow fibers 211 are configured to filter fluid by forcibly conducting fluid across the hollow fibers 211. Any number of semi-permeable hollow fibers 211 that are commercially available for this intended purpose may be used. For example, semi-permeable hollow fibers 211 come in variety of dimensions and can be formed of polymers, such as polysulfone, or be cellulose-based.

The housing 210 includes a number of different integral ports that permit fluid to enter and exit the housing 210. The housing 210 includes a first end 212 and a second end 214. At the first end 212 there is a first port 213 that can be in the form of an inlet and at the second end 214, there is a second port 215 that can be in the form of an outlet. The housing 210 further includes a third port 216 that can be located along the side of the housing 210 (FIG. 2) and optionally and according to some housing constructions, the housing 210 can include a fourth port 217 (FIG. 2). The third and fourth ports 216, 217 are in fluid communication with the hollow interior of the housing 210 that is located external to and about the hollow fibers 211. In contrast, the first and second ports 213, 215 are in fluid communication with opposing ends of the fibers 211 and more specifically are in fluid communication with the lumens thereof. Header spaces 227, 229 can be formed at the ends of the cartridge and as is known in the art, a potting compound 225 can be used to seal around the fibers 211 at the ends of the housing, while leaving the lumens of the fibers 211 in fluid communication with the open header spacers. The first port 213 is thus in fluid communication with the first header space 227 and the second port 215 is in fluid communication with the second header space 229.

In the illustrated embodiment, the first port 213 represents the inlet port for injecting fluid samples and reagents into the lumens of the fibers 211 for filtering. Thus, the first conduit 12 (which can include the peristaltic pump section when a peristaltic pump is used) is connected at one to the first port 213. Pump 210 is shown in FIG. 2 as well.

In certain embodiments, commercially available filtration device may be used to filter the fluid sample. Examples of commercially available filtration device include High Performance Antipyrogenic Ultrafilter for Replacement Solutions (D150/U) from Medica Group. (See M27053 rev.02 modifica ME190314C del 19.03.2014).

In certain embodiments, the filtration device 200 has a fiber membrane (fibers 211) with pore size from about 0.002 micron to about 0.01 micron.

In certain embodiments, the filtration device 200 has a filtration capacity to reduce the volume of an initial fluid sample on the order of as much as a 50 times reduction in sample volume.

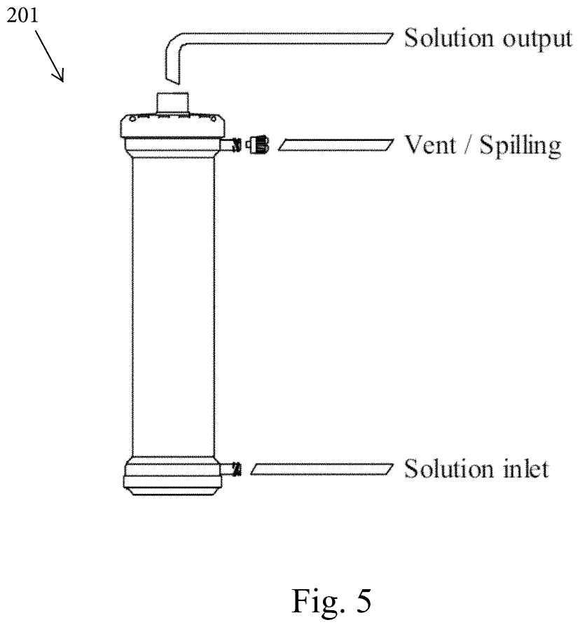

The fourth port 217 can be closed with a cap (not shown), while the third port 216 can be connected to a third conduit 16 that receives purified fluid (i.e., fluid that has been filtered across the fibers 211) and delivers the purified fluid to either a collection vessel 301, to a drain, or even can be open at its distal ends to deliver the purified fluid back to ground soil if the system 10 is being operated outside. Alternatively, the third port 216 can be capped and the third conduit 16 is connected to the fourth port 217. In the embodiment shown in FIG. 4, the cartridge only includes a third port 216 and thus, the third conduit 16 is connected to the third port 216. FIG. 5 illustrates another filtration device 201 that can be used. In this case (FIG. 5), the solution to be filtered is delivered into a side port that is in communication with the space around the fibers and the solution is outputted at the end of the cartridge by being conducted across the fibers into the lumens and then flowing to the open header space at the end, and venting is along a side port as shown. The operation of this cartridge is similar to the others described herein in that the target pathogen is collected as filtrate within the lumens of the fibers.

The third port 216 or the fourth port 217 where present can thus be considered to be a fluid outlet port.

The second port 215 acts as a venting port (air purge) and is connected to a second conduit 14 which can have a clamp 219 along its length for selectively closing off the second conduit 14 and also has an air valve (air check) or the like to permit air to be vented from inside the cartridge and more particularly from within the lumens. When the clamp is open, venting is permitted. It will be appreciated that the fibers 211 are initially filled with air and need to be wetted (primed) with fluid. As fluid (liquid) is delivered into the first port 213 into the lumens of the fibers 211, the air within the lumens is expelled downward to the second port 215 where it is vented. The venting mechanism in the second conduit 14 is designed so that no liquid is expelled through the second port 215 into the second conduit 14. From the second conduit 14, air is vented to atmosphere.

In operation, the system 10 is first operated to generate filtrate within the lumens of the fibers 211 by delivering liquid from the source 100 into the lumens of the fibers 211 using pump 210 or another mechanism. After the air within the lumens is purged through the second port 215. The liquid (e.g., water) is conducted across the fibers 211 and purified water exits through the third port 216 (or fourth port 217). Filtrate which comprises any target pathogens is left behind within the lumens of the fibers 211. Once a sufficient amount of liquid is filtered through the filtration device 200, the delivery of the liquid is stopped. A sufficient amount of liquid could be 1 liter of liquid, or between 200 milliliters to 2 liters depending on the sampling protocol, from standard potable water sources, or could be 10 liters of liquid, or between 1 liter and 100 liters depending on the sampling protocol, from ultrapure water sources such as in the dialysis or pharmaceutical manufacturing industries.

For example, the first conduit 12 can be removed from the source 100. The next step can be to run air through the fibers 211 of the filtration device 200 as by using the pump 210. Running air through the first port 213 into the fibers 211 causes any liquid within the fibers 211 to be conducted across the fibers 211 and also reserves to dry the lumens which results in the bacteria being present as a residue that is within the lumens along the walls of the fibers 211. The delivered air is vented through the second port 215.

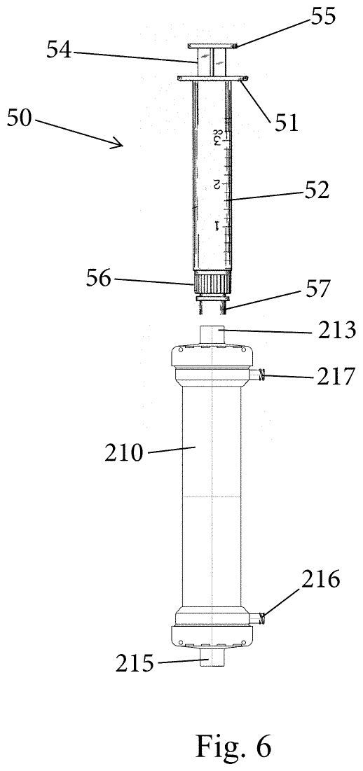

Once this step is performed, the filtrate (in residue form) is then processed and collected by treating the filtrate to lyse the bacteria nucleus therein and collect the lysed bacteria (FIG. 6). This step can be performed by disconnecting the first conduit 12 from the first port 213 and then connecting a device for delivering a liquid into the lumens of the fibers to contact the filtrate. For example, and as shown in FIG. 6, a syringe 50 can be used (for simplicity, the conduits are not shown connected to the cartridge in FIG. 6). Syringe 50 can be a conventional syringe that includes a barrel 52 and a slidable plunger 54 that is inserted into the barrel 52. A flange 51 extends from the barrel 52 for holding the barrel 52 and a flange 55 extends from the plunger 54. At a distal end of the barrel 52, a first connector 56 is present and can mate with a connector part 57, such as a Luer lock, that is configured to connect to the first port 213 in a sealed manner.

Initially, the barrel 52 contains the liquid that is used to lyse the target pathogen(s). 20 milliliters of lysis buffer, or between 5 milliliters and 100 milliliters depending on the filter, will overfill the volume of inner lumen of the fiber in the filter. The barrel 52 is fluidly connected to the first port 213 and then the plunger 54 is manipulated to deliver (expel) the liquid into the lumens of the fibers 211 for contacting the bacteria (filtrate/residue). The plunger 54 preferably is slowly moved to ensure a slow delivery of the liquid to allow for proper lysing of the pathogens.

In certain embodiments, this lysis treatment involves a lysis buffer comprising: 4.5 M GITC (guanidinium isothiocyanate) dissolved in Tris(10 mM)-EDTA (1 mM) (TE) buffer (pH 8.0) polyadenylic acid [poly(A)] (17.6 μg/mL); 0.14 M sodium acetate (NaOAc); 0.24 M NaCl; 0.4% sodium sulphite; 0.2% dithioerythritol (DTE); 0.02% Sodium dodecyl sulfate (SDS); and 0.4% Tween 20.

In certain embodiments, lysis buffer is slowly inserted to the filtration device 200 manually via the syringe 50 to absorb the filtrate, over the time period of about 5 to 60 seconds. More than 20 milliliters of lysis buffer, or between 5 milliliters and 100 milliliters depending on the filter, is extracted from the filtration device.

Once the lysis treatment is completed, the user then moves the plunger 54 in the opposite direction so as to pull the solution within the lumens of the fibers 211 back into the barrel 52 of the syringe 50 so as to collect the lysed pathogen solution (i.e., lysed bacterial solution) within the barrel 52.

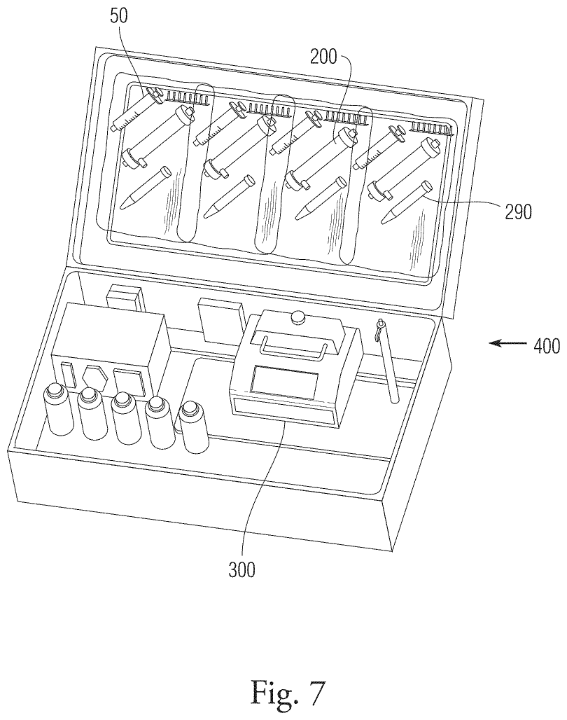

Next, the lysed bacterial solution can be placed into a container or vessel by pushing the plunger 54 forward to expel the lysed bacterial solution into the container. The container can be of a type that mates with the connector 57. The container can be in the form of a reservoir tube 290 shown in FIG. 7.

Next, a prescribed amount of lysed bacterial solution is removed from the container (reservoir tube) and delivered into a well or the like that is part of the molecular detection device 300. For example, an amount of between about 10 microliters and 50 microliters can be aliquoted from the container and then delivered into the one or more test wells of the molecular detection device 300. The wells of the molecular detection device 300 contain lyophilized primers, cap oligos, probes and master mix for polymerase chain reaction detection and are reconstituted with solution. The wells are individually identified with indicia so that the user knows which wells are to be used. The primers, probes and reagents, that are described herein, all come in solution form.

In contrast to conventional lyophilization techniques, the lyophilization step of the present method involves the lyophilization of the primers, probes and reagents. As a result, and in contrast to conventional techniques, additional consideration and calculation are required to ensure refinement of the reagent ratios and sample volumes (in conventional processing, these considerations are not required).

As will further be understood, the lysed water sample itself is used to reconstitute the lyophilized reagents, primers and probes.

The molecular detection device 300 incubates the lysed bacterial solution under amplification conditions with oligonucleotide primers and DNA polymerase; and is configured to amplify a specific DNA nucleotide sequence that is generally known to be ubiquitous to most all Gram-negative bacteria.

Any number of suitable molecular detection devices 300 can be used and preferably, as discussed herein, the molecular detection device 300 preferably has a small footprint and is portable. In some embodiments, the device 300 can be battery powered.

It will be understood that the molecular detection device 300 can be made up of a number of individual pieces of equipment that perform different operations that are described herein. For example, the molecular detection device, and its related equipment, can be configured to receive a portion of the lysed bacterial solution and perform an amplification process to produce an amplified bacterial solution that can then quantify the Gram-negative bacteria DNA of the amplified bacterial solution. Next, the molecular detection device 300 is also configured to estimate the endotoxin-producing bacteria level of the water sample from the analysis of the bacterial data.

Computing Device

As described herein, the molecular detection device 300, as well as other components of the system 10, can be part of a computing device. For example, The process controller (processor) 305 of the molecular detection device 300 that includes various hardware and software components that serve to enable operation of the system 10, including the processor and memory 310 and can include an interface, display, storage and a communication interface. The processor controller (processor) 305 serves to execute software instructions that can be loaded into memory 310. Process controller (processor) 305 can be one or more of processors, a multi-processor core, or some other type of processor, depending on the particular implementation.

One or more software modules can be in storage and/or memory 310. The software modules can include one or more software programs or applications having computer program code or instructions to be executed in processor. Such computer program code or instructions for carrying out operations for aspects of the systems and methods disclosed herein and can be written in any combination of one or more programming languages. The program code can execute entirely on process controller 305, as a stand-alone software package, partly on process controller, or entirely on another computing/device or partly on another remote computing/device. In one or more embodiments, a remote computing device can be connected to process controller 305 through any type of direct electronic connection or network, including a local area network (LAN) or a wide area network (WAN), or the connection can be made to an external computer (for example, through the Internet using an Internet Service Provider). The communication interface is also operatively connected to the processor and can be any interface that enables communication between the process controller and various devices, machines and/or elements including, but not limited to robot, imaging device, etch controller, clean controllers, chemistry controllers, etc. Preferably, communication interface includes, but is not limited to, Ethernet, IEEE 1394, parallel, PS/2, Serial, USB, VGA, DVI, SCSI, HDMI, a Network Interface Card (NIC), an integrated network interface, a radio frequency transmitter/receiver (e.g., Bluetooth, cellular, NFC), a satellite communication transmitter/receiver, an infrared port, and/or any other such interfaces for connecting process controller to other devices and/or communication networks, such as private networks and the public networks (e.g., Internet). Such connections can include a wired connection (e.g. using the RS232 or other standard) or a wireless connection (e.g. using the 802.11 or other standard). It is to be understood that communication interface can be practically any interface that enables communication to/from the process controller.

Molecular Detection Device 300—PCR

Amplification of the target DNA sequence is by means of selected primer pairs according to a procedure known as Polymerase Chain Reaction, hereinafter referred to simply as PCR. PCR amplification of nucleotide sequences is described in U.S. Pat. No. 4,683,202, the disclosure of which is incorporated herein by reference. The PCR amplification process comprises amplifying a selected or targeted nucleic acid sequence by treating the two separate complementary strands of the nucleic acid sequence with two oligonucleotide primers, each being complementary to one of the two strands, to anneal the primers to their complementary strands, then synthesizing extension products of said primers by polymerase to extend said primers to make fully double-stranded replicas of the selected target nucleic acid sequence, followed by separation (denaturation) of the extension products and repeating this amplification sequence the desired number of cycles to increase the concentration of the selected nucleic acid sequence. The process is utilized for amplification and detection of DNA fragments in a sample. In one particular implementation, the primer is encoded by a nucleic acid sequence of SEQ ID 1 or 2. In a further implementation, the probe is encoded by a nucleic acid sequence of SEQ ID 3. One of skill in the art would understand that some bases can be deleted from or added to the end of SEQ ID NOs: 1 and 2 and said primers can still amplify the nucleic acid. One of skill in the art would understand that some bases can be deleted from or added to the end of SEQ ID No. 3 and said probe can still detect the nucleic acid. Accordingly, this disclosure includes primers wherein some bases are deleted or added to sequences of SEQ ID NOs: 1, 2 or 3. In a further implementation, a primer is provided having a nucleic acid sequence that is at least 90% similar to SEQ ID NO:1 or SEQ ID 2. In yet a further implementation, a probe is provided having a nucleic acid sequence that is at least 90% similar to SEQ ID NO:3

In one embodiment, described herein is a method of analysis of a fluid sample to detect the presence of target pathogens or indicator microorganisms using a molecular detection device, comprising steps of: preparing test wells containing lyophilized primers, cap oligos, probes and master mix for polymerase chain reaction detection; placing test wells into a multi-well, multi-channel thermocycling device and running them on repeated heat cycle pattern; calculating the concentration of colony or plaque forming units in each test sample, using quantification of nucleic acid in fluorescence units for the DNA fragments target pathogen.

In certain embodiment, the first heating cycle is run for between 30 and 180 seconds longer than the standard heating cycles to further break-up potential cellular walls and free DNA fragments.

In certain embodiment, subsequent cycles are standard heating cycles for up to 40 cycles to facilitate the polymerase chain reactions to amplify and detect the nucleic acid fragments of interest.

Quantification of Total Bacteria

Described herein is a method of quantifying the number of nucleic acid segments in fluorescence units for each target DNA fragment to calculate the overall concentration of bacteria in each test sample. This total concentration would include all of DNA segments from all of the bacteria present in the fluid sample, regardless of bacterial genus.

The concentration of total bacteria is determined by the number of nucleic acid molecules of the assay target DNA fragment present in the polymerase chain reaction volume. The number of nucleic acid molecules present is equal to

N₀=Nₜ/(E+1)^{Ct}

where Cₜ is the fractional threshold cycle as determined by the fluorescent signal above baseline, Nₜ is the number of amplicon molecules at fluorescent threshold, and E is amplification efficiency, compared to replicate standard curves for the assay. The concentration of nucleic acid is equal to

Aₙₐ=N₀/Vₚ
where Aₙₐ is the concentration of nucleic acid and Vₚ is the polymerase chain reaction volume.

The corresponding concentration of colony or plaque forming units is determined by the number of copies of the assay target nucleic acid normally present in the viable target pathogen and is equal to A_{fu}=Aₙₐ/A_{ba} where A_{fu} is the concentration of colony forming units in the polymerase chain reaction volume in microliters and A_{ba} is the average or normal number of copies of the assay target nucleic acid segments present.

For the A_{fu} value to be practically useful and meaningful to scientific, regulatory, and public health standards for the presence of bacteria in a fluid sample (i.e., water), it remains to calculate the concentration of estimated target bacteria colony or plaque forming units per milliliter for comparison. The concentration of total bacteria colony or plaque forming units per milliliter in the original fluid sample taken is equal to:

Aₛₐₘₚₗₑ=(A_{fu}*V_{con}*10³)/V_{orig}

where V_{con} is the volume of the concentrated lysate solution collected in milliliters and V_{orig} is the volume of the fluid sample collected in milliliters.

Aₛₐₘₚₗₑ represents the total number of bacterial colony or plaque forming units. The total number of then multiplied by a percentage of each bacterial family counted in the total sample to determine the concentration of any given bacterial family in the sample (A_{BF1}, A_{BF2}, etc.). The percentage of each bacterial genus is determined in a later step using the genetic sequencing device.

Estimation of Endotoxin

The resulting total count for each Gram-negative bacteria can be used to estimate the total endotoxin by multiplying the count by the average lipopolysaccharide content of Gram-negative bacterial cell walls.

Fluid Analysis Kit

Described herein is a kit for use in a process for analyzing fluid sample the presence of bacteria, comprising; the filtration device 200, collection conduit 12, peristaltic pump device 210, dispensing third conduit 16, the molecular detection device 300, and the genetic sequencing device 350.

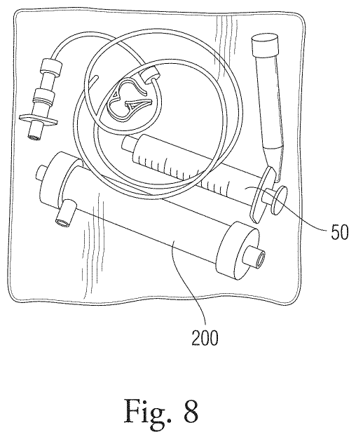

FIG. 7 shows a kit 400 that includes an openable portable case that contains the components of the system 10. The molecular detection device 300 and genetic sequencing device 350 can be provided along a floor of the case. Plural portable filtration devices 200 can be mounted along a lid of the case along with other components such as syringes 50 and containers 290. Tubing and the like can also be provided in the case along with pump 210 which can be battery driven to allow portable use outside of where electricity is available. The molecular detection device 300 can also be battery powered. FIG. 8 shows an individual vacuum sealed package that contains certain components, such as one filtration device 200 and one syringe 50 and can include one container 290. By being prepackaged, the user can simply take one package to perform one test of one sample. To perform a second test of a second sample, another package is accessed.

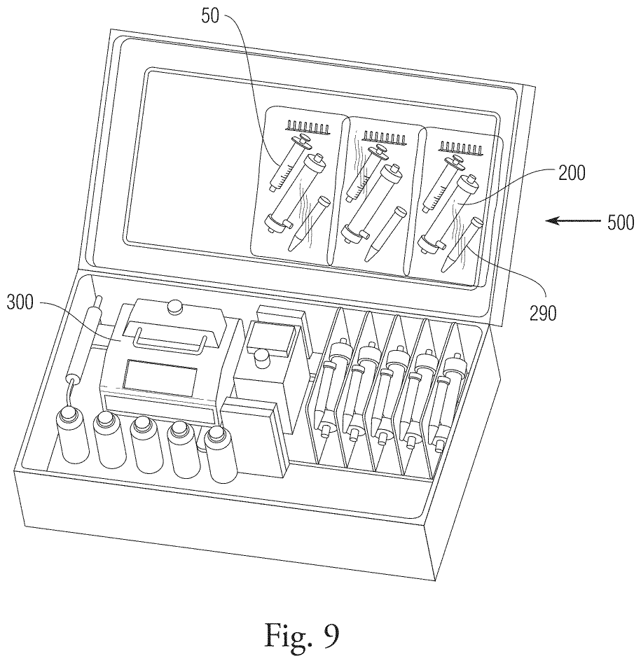

FIG. 9 shows another kit 500 similar to the kit 400 and includes components 200, 50, 290 of the system and can include all other components, such as the conduits (tubing), etc. In FIG. 9, there can be divider walls to hold and separate the components (consumables).

In one embodiment, the kit comprises an outside carrier with a hard surface (e.g., plastic case). In one embodiment, the kit is portable and the case can include a handle on its openable lid/cover. In certain embodiments, commercially available outside carrier may be used. Examples of commercially available outside carrier include 1615 Air Case from Pelican. (See www.pelican.com/us/en/product/cases/air/1615)

In one embodiment, the kit includes a hardware mobile electronic device and a software application that provide a graphical user interface for human interaction, comprising information inputs and reporting outputs.

Mobile Analysis Device and Application

Described herein is a hardware mobile electronic device and a software application that provide a graphical user interface for human interaction, comprising information inputs and reporting outputs. In one embodiment, the mobile electronic device and the application communicate wirelessly with the molecular detection device 300 as well as a web-based data storage and analytic computing capability. In one embodiment, the mobile electronic device 300 and the application wirelessly transmit the programming of the polymerase chain reaction thermocycling device (device 300), collect and associate sample identification and associated metadata, wirelessly retrieve the results of the polymerase chain reaction analysis from the thermocycling device and the genetic sequencing device, transmit the results to a web-based computer platform to perform the calculations to determine the bacteria colony or plaque forming units per milliliter in the original fluid sample and to determine the percentage of different bacterial families counted in the sample, and to generate the result reports for printing or electronic dissemination.

In this regard and as already described herein, the molecular detection device 300 include respective processor 305, that is configured to control the various components of the devices 300, 350 and carry out aspects of the systems and methods disclosed herein. The microprocessor can be a number of processors, a multi-processor core, or some other type of processor, depending on the particular implementation. In some implementations the microprocessor is configured by executing one or more software modules that can be loaded into a memory and executed by the microprocessor. The one or more software modules can comprise one or more software programs or applications having computer program code or a set of instructions executed in the microprocessor. Such computer program code or instructions can be written in any combination of one or more programming languages. Preferably, included among the software modules are a user input module, a display module, a stimuli control module and a communication module. During execution of the software modules, the microprocessor configures the device 300 to perform various operations described herein.

Memory can be, for example, a random access memory (RAM) or any other suitable volatile or non-volatile computer readable storage medium. In addition, memory can be fixed or removable and can contain one or more components or devices such as a hard drive, a flash memory, a rewritable optical disk, a rewritable magnetic tape, or some combination of the above. In addition, memory can be onboard the microprocessor. In addition, it should be noted that other information and/or data relevant to the operation of the present systems and methods can also be stored on memory, as will be discussed in greater detail below.

A display (e.g., LCD display) can also be operatively connected to the microprocessor. The display can be a digital display such as a segment display, a dot matrix display or a 2-dimensional display and can incorporate, by way of example and not limitation, a liquid crystal display, light emitting diode display, electroluminescent display and the like. The display provides an output to the user of information relevant to the operation of the device 300.

A control button and touch interface represent one or more user input devices that are operatively connected to the microprocessor. Such user input devices serve to facilitate the capture commands from the user such as an on-off commands and operating parameters related to the operation of the device. User input devices can also serve to facilitate the capture of other information from the user and provide the information to the microprocessor.

The control button can be one or more switch(es), button(s), knob(s), key(s). The touch interface is a touch sensitive device that can be is placed in register on the top of the display or on/around the perimeter of the display or anywhere on the housing. A touch interface is comprised of one or more thin, transparent layers that can detect when and where a user touches the interface and it allows a user to interact directly with what is displayed without requiring an intermediate device such as a computer mouse. The touch interface can be constructed using, by way of example and not limited to, resistive, capacitive, acoustic, infrared, optical imaging, or dispersive signal technology.

By way of further example, the touch interface and display can be integrated into a touch screen display. Accordingly, the screen is used to show a graphical user interface, which can display various fields or virtual buttons that allow for the entry of information by the user. Touching the touch screen at locations corresponding to the display of a graphical user interface allows the person to interact with the device to enter data, change settings, control functions, etc. So, when the touch screen is touched, interface communicates this change to microprocessor, and settings can be changed or user entered information can be captured and stored in the memory.

The communication interface (discussed previously) can also be operatively connected to the microprocessor. The communication interface can be any interface that enables communication between the device 300 and external devices, machines and/or elements including a user's computer system. Communication interface can include but is not limited to a Bluetooth, or cellular transceiver, a radio transceiver, an NFC transceiver, a satellite communication transmitter/receiver, an optical port and/or any other such interfaces for wirelessly connecting the device 300 to an external computing device, such as a tablet, laptop, etc.

It can be appreciated that aspects of the present systems and methods can take the form of an entirely hardware embodiment, an entirely software embodiment (including firmware, resident software, micro-code, etc.), or an embodiment combining software and hardware. One of skill in the art can appreciate that a software process can be transformed into an equivalent hardware structure, and a hardware structure can itself be transformed into an equivalent software process. Thus, the selection of a hardware implementation versus a software implementation is one of design choice and left to the implementer. For example, the microcontroller can take the form of a circuit system, an application specific integrated circuit (ASIC), a programmable logic device, or some other suitable type of hardware configured to perform a number of operations. With a programmable logic device, the device is configured to perform the number of operations. The device can be reconfigured at a later time or can be permanently configured to perform the number of operations. Examples of programmable logic devices include, for example, a programmable logic array, programmable array logic, a field programmable logic array, a field programmable gate array, and other suitable hardware devices. With this type of implementation, software modules can be omitted because the processes for the different embodiments are implemented in a hardware unit.

### EXAMPLES

The present disclosure may be better understood by reference to the following non-limiting example, which is presented in order to more fully illustrate the preferred embodiments of the disclosure. They should in no way be construed to limit the broad scope of the disclosure.

qPCR of Gram Negative Specific Region of the 16S rRNA Gene (Prophetic)

PCR amplification can be performed using a New England Biolabs LongAmp® Taq 2X Master Mix (Cat #M0287S) (or other suitable kit). The PCR solution contains a master mix provided in Table 2.

TABLE 2
Master Mix. Volume of sample: 5-10 ul for a final
reaction (Master Mix + sample) volume of 25 ul.

| | | Volume for 1X |
|---|---|---|
| Reagents | [Final] | (ul) |
| dNTP mix (10 mM) | 400 uM | 1 |
| Target A Primer/Probe Mix | 0.5 uM/0.3 uM | 1 |
| Target B Primer/Probe Mix | 0.5 uM/0.3 uM | 1 |
| LongAmp taq2x Master Mix | | 12.5 |
| dH2O | — | 6-11 |

| | |
|---|---|
| Total Volume of Master Mix | 15-20 |

A region of 16S rRNA gene is amplified using primer/probe provided in Table 3 for each target.

TABLE 3
Primers and Probe

| | | |
|---|---|---|
| Seq. ID No. 1 | Forward | ATGACGTCAAGTCATCATGG |
| | Primer | |
| Seq. ID No. 2 | Reverse | AGGAGGTGATCCARCCGCA |
| | Primer | |
| Seq. ID No. 3 | Probe | CACCGCCCGTCACACCATGGGA |

TABLE 4
Panel Fluorophores

| Target | Fluorophore | Excitation (nm) | Emission (nm) |
|---|---|---|---|
| Target 1 | FAM | 495 | 520 |
| Target 2 | HEX | 538 | 555 |

A Primer Mix is prepared according to Table 5.

TABLE 5
Primer Mix

| Reagents | Final concentration | |
|---|---|---|
| Forward Primer | 13.3 | uM |
| Reverse Primer | 13.3 | uM |
| 1X TE (pH 8.0) | Variable | |
| Total Volume | 100 | ul |

15-20 μL of the master mix is placed into the appropriate number of qPCR tube(s). 5-10 μL of sample DNA/RNA (1 pg-1 ng) or RNase-free water (for no template control reactions) is added into each qPCR tube.

The tubes are inserted into a real time thermocycler with the cycling conditions provided in Table 6. In certain embodiment, the ramp rate is 3° C./second.

TABLE 6
Cycling conditions

| | | |
|---|---|---|
| Cycling Conditions | Time | Cycles |

| | | | |
|---|---|---|---|
| 95° C. | 1 | min | |
| 95° C. | 20 | sec | 35X |
| 55° C. | 30 | sec | |
| 65° C. | 2 | min | |

Comparisons with Third Party Assays

It has been determined that the portable system 10 described herein provides an improved approach for the detection of Gram-Negative bacteria within a liquid sample. For example, the portable system 10 has a Sensitivity of 0.001 EU/ml, which represents an improvement over the portable assay kits and systems available in the art.

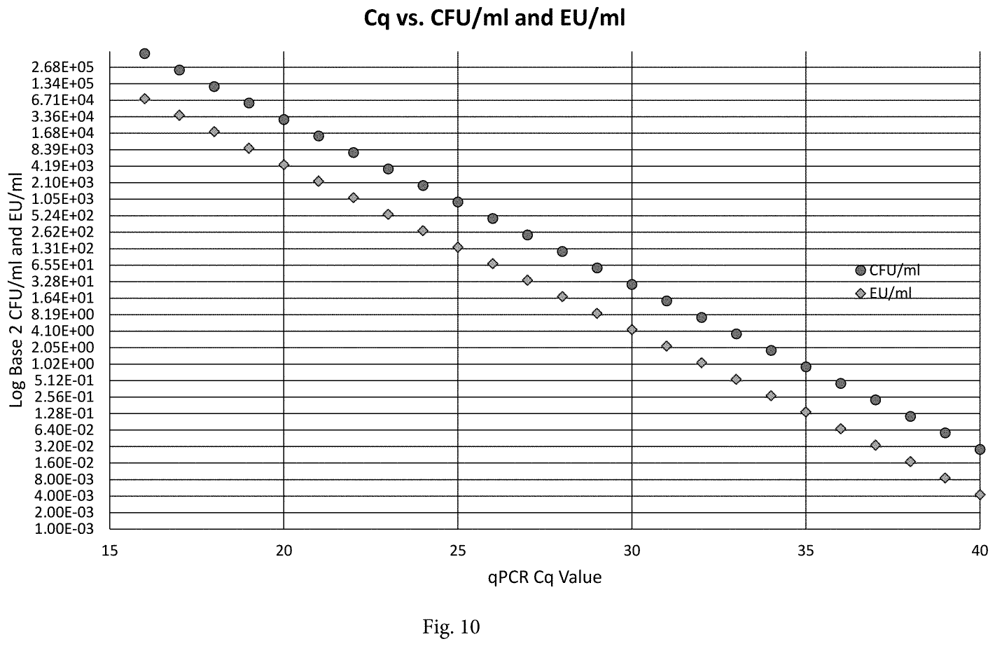

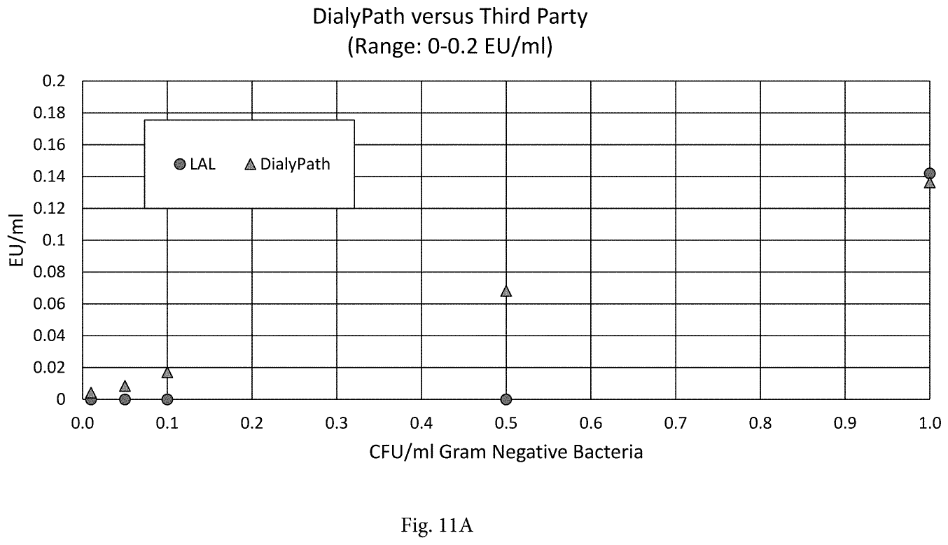

As shown with particular reference to FIG. 10, a graph is provided detailing Cq versus CFU/ml and EU/ml of endotoxin of serial dilutions of E. coli in water analyzed using the qPCR assay for Gram-negative bacteria.

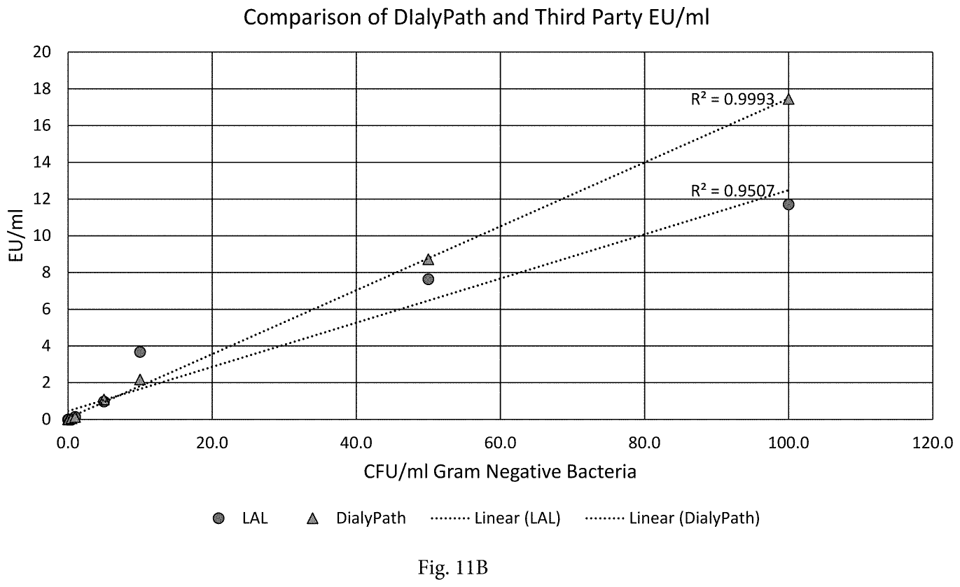

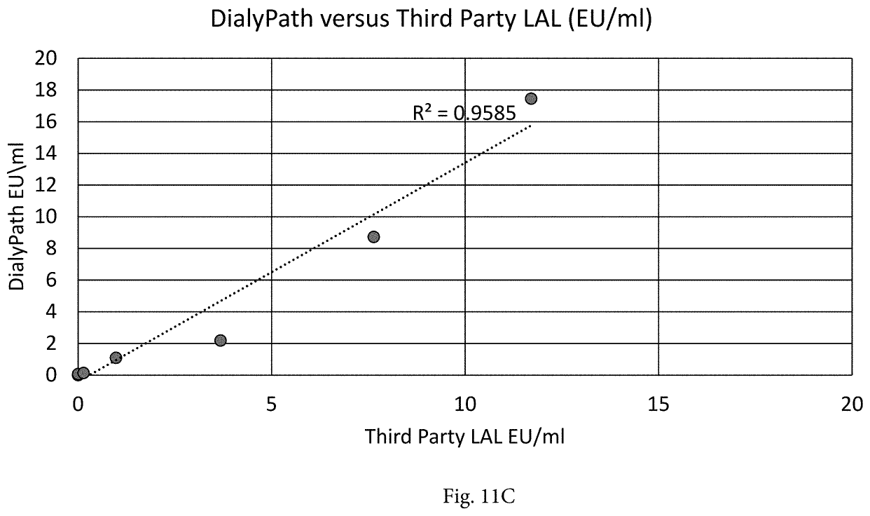

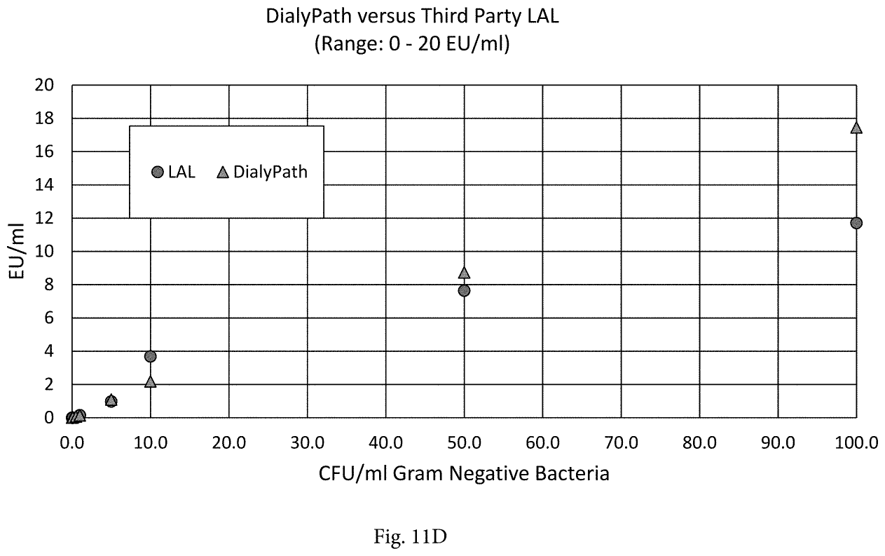

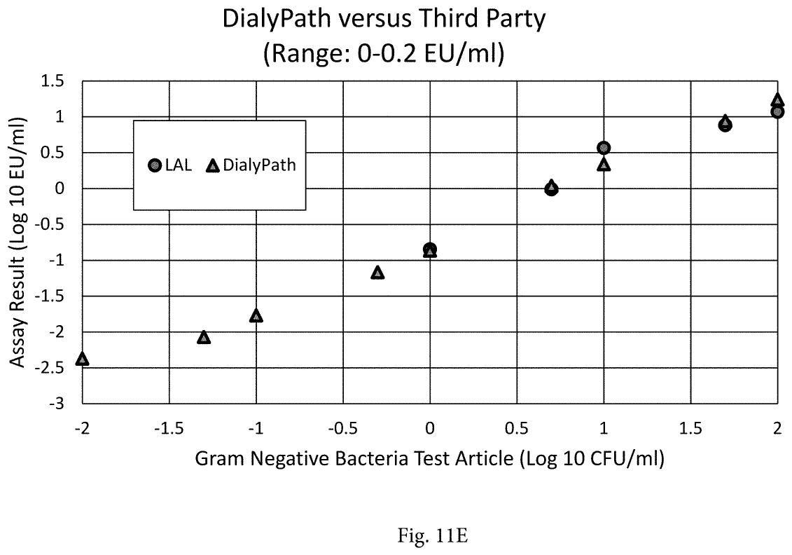

As shown with particular reference to FIGS. 11A-E, scatter plots are provided detailing the performance of an implementation of the portable system 10 using qPCR for the Gram-negative bacteria (referred to herein as the “DialyPath”) relative to an alternative assay test (referred to as “LAL”). As shown, FIGS. 11A and 11B provide comparisons of the performance of the portable system 10 relative to an alternative array, where the plot provides axis for CFU/ml and EU/ml of endotoxin. FIGS. 11C-11E compare the EU/ml results using the approaches described herein compared to the alternative assay approach. As shown in the provided figures, the approach described herein provides improved measurements of EU/ml within a sample relative to alternative approaches evaluated.

Notably, the figures and examples above are not meant to limit the scope of the present invention to a single embodiment, as other embodiments are possible by way of interchange of some or all of the described or illustrated elements. Moreover, where certain elements of the present invention can be partially or fully implemented using known components, only those portions of such known components that are necessary for an understanding of the present invention are described, and detailed descriptions of other portions of such known components are omitted so as not to obscure the invention. In the present specification, an embodiment showing a singular component should not necessarily be limited to other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present invention encompasses present and future known equivalents to the known components referred to herein by way of illustration.

The foregoing description of the specific embodiments will so fully reveal the general nature of the invention that others can, by applying knowledge within the skill of the relevant art(s) (including the contents of the documents cited and incorporated by reference herein), readily modify and/or adapt for various applications such specific embodiments, without undue experimentation, without departing from the general concept of the present invention. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

While operations are depicted in the drawings in a particular order, this should not be understood as requiring that such operations be performed in the particular order shown or in sequential order, or that all illustrated operations be performed, to achieve desirable results. In certain circumstances, multitasking and parallel processing can be advantageous. Moreover, the separation of various system components in the embodiments described above should not be understood as requiring such separation in all embodiments, and it should be understood that the described program components and systems can generally be integrated together in a single software product or packaged into multiple software products.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components, and/or groups thereof.

It should be noted that use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having the same name (but for use of the ordinal term) to distinguish the claim elements. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Particular embodiments of the subject matter described in this specification have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain embodiments, multitasking and parallel processing can be advantageous.

Publications and references to known registered marks representing various systems cited throughout this application are incorporated by reference herein. Citation of any above publications or documents is not intended as an admission that any of the foregoing is pertinent prior art, nor does it constitute any admission as to the contents or date of these publications or documents. All references cited herein are incorporated by reference to the same extent as if each individual publication and references were specifically and individually indicated to be incorporated by reference.

While various embodiments of the present invention have been described above, it should be understood that they have been presented by way of example, and not limitation. It would be apparent to one skilled in the relevant art(s) that various changes in form and detail could be made therein without departing from the spirit and scope of the invention. Thus, the present invention should not be limited by any of the above-described exemplary embodiments, but should be defined only in accordance with the following claims and their equivalents.

What is claimed is:

1. A method for rapidly isolating and detecting waterborne bacteria within a liquid sample comprising the steps of:
filtering the liquid sample using a portable filtration device so that the waterborne bacteria forms a filtrate on a surface of a filter of the portable filtration device and purified liquid that results from the liquid sample being conducted across the filter is discharged from the portable filtration device;
introducing air into the portable filtration device so that the filtrate forms a residue on the surface of the filter and the liquid sample is at least substantially expelled from the portable filtration device;
using a fluid delivery device to introduce a lysing agent into the portable filtration device resulting in lysing of the waterborne bacteria and formation of a lysed bacterial solution;
using the fluid delivery device to remove an amount of the lysed bacterial solution; and
introducing at least a portion of the removed amount of lysed bacterial solution into a molecular detection and amplification device that is configured to detect a presence of bacteria within the amplified bacterial solution;
and detecting the presence of bacteria in the amplified bacterial solution.

2. The method of claim 1, wherein the step of filtering the liquid sample using the portable filtration device acts to concentrate the waterborne bacteria as the filtrate formed within the filter.

3. The method of claim 1, wherein the portable filtration device comprises a cartridge and the filter comprises a plurality of semi-permeable filtering elements; an inlet port for delivering the liquid sample into lumens of the plurality of semi-permeable filtering elements; an outlet port for discharging the purified liquid; and a vent port for venting air from the lumens of the plurality of semi-permeable filtering elements.

4. The method of claim 3, wherein the plurality of semi-permeable filtering elements comprises hollow fibers, with the inlet port being in fluid communication with first ends of the hollow fibers and the vent port being in fluid communication with second ends of the hollow fibers, the outlet port being in fluid communication with a hollow space surrounding the plurality of semi-permeable filtering elements.

5. The method of claim 3, wherein the step of filtering the liquid sample includes the step of pumping the liquid sample into the lumens of the plurality of semi-permeable filtering elements.

6. The method of claim 3, wherein the step of introducing air comprises delivering air through the inlet port into the lumens of the plurality of semi-permeable filtering elements until the filtrate forms the residue and the liquid is at least substantially expelled.

7. The method of claim 1, wherein the fluid delivery device comprises a syringe that is connected to the inlet and is operated by moving a plunger within a barrel in a first direction to deliver the lysing agent into the filter into contact with the residue such that the lysing agent absorbs the residue and forms the lysed bacterial solution.

8. The method of claim 7, wherein the step of using the fluid delivery device to remove the amount of the lysed bacterial solution comprises moving the plunger within the barrel in a second direction to draw the amount of lysed bacterial solution into the barrel.

9. The method of claim 3, wherein the lysing agent is delivered into the lumens of the semi-permeable filtering elements.

10. The method of claim 9, wherein the lysing agent comprises a lysis buffer.

11. The method of claim 10, wherein the lysis buffer comprises: 4.5 M GITC (guanidinium isothiocyanate) dissolved in Tris(10 mM)-EDTA (1 mM) (TE) buffer (pH 8.0) polyadenylic acid [poly(A)] (17.6 μg/mL); 0.14 M sodium acetate (NaOAc); 0.24 M NaCl; 0.4% sodium sulphite; 0.2% dithioerythritol (DTE); 0.02% Sodium dodecyl sulfate (SDS); and 0.4% Tween 20.

12. The method of claim 1, wherein the portion of the removed amount of lysed bacterial solution comprises between 10 microliters and 50 microliters of solution.

13. The method of claim 1, wherein the portion of the removed amount of lysed bacterial solution is delivered into the one or more test wells of the molecular detection device, each well containing lyophilized primers, cap oligos, probes and master mix for polymerase chain reaction detection and are reconstituted with another solution.

14. The method of claim 1, wherein the molecular detection device is configured to incubate the lysed bacterial solution under amplification conditions with oligonucleotide primers and DNA polymerase and is configured to detect amplified target DNA to determine the presence or absence in the lysed bacterial solution or indicator microorganisms carrying selected target DNA nucleotide sequence.

15. The method of claim 1, wherein the liquid sample has a volume between about five-hundred milliliters to about one liter.

16. The method of claim 14, wherein the oligonucleotide primers have a sequence of SEQ ID 1 or 2.

17. A method for rapidly isolating and detecting waterborne pathogens, such as waterborne bacteria, within a liquid sample comprising the steps of:
filtering the liquid sample using a portable filtration device so that the waterborne pathogens forms a concentrated filtrate that comprises the waterborne pathogens and is contained within the portable filtration device;
introducing a lysing agent into the portable filtration device resulting in lysing of the waterborne pathogens and formation of a lysed waterborne pathogen solution that is contained within the portable filtration device;
removing an amount of the lysed waterborne pathogen solution from the portable filtration device; and
introducing at least a portion of the removed amount of lysed waterborne pathogen solution into a molecular detection and amplification device that is configured to detect whether waterborne pathogens, such as bacteria, are present in the amplified liquid sample.

18. The method of claim 17, wherein the waterborne pathogens comprise bacteria.

19. A portable system for rapidly detecting waterborne pathogens, such as bacteria, comprising:
a portable filtration device including a first port for receiving a liquid sample to be tested; a second port for venting air and a third port for discharging purified liquid, the portable filtration device including a plurality of semi-permeable filtering elements for filtering the liquid sample and generating a filtrate within the semi-permeable filtering elements, the filtrate containing the waterborne pathogens;
a delivery device configured for being sealingly mated to the first port and configured to deliver a lysing agent into lumens of the plurality of semi-permeable filtering elements for lysing of the waterborne pathogens and formation of a lysed waterborne pathogen solution; and
a molecular detection and amplification device for analyzing the lysed waterborne pathogen solution and detecting whether waterborne pathogens are present in the amplified, lysed waterborne pathogen solution.

20. The system of claim 19, wherein the plurality of filtering elements comprises a plurality of hollow fibers having a pore size from about 0.002 micron to about 0.01 micron.

21. The system of claim 19, wherein the portable filtration device has a filtration capacity to reduce the volume of the liquid by a factor of up to 50 times.

22. The system of claim 19, wherein the molecular detection device includes test wells and the molecular detection device is configured to incubate the lysed waterborne pathogen solution under amplification conditions with oligonucleotide primers and DNA polymerase; and detect amplified target DNA to determine the presence or absence in the liquid sample of waterborne pathogens or indicator microorganisms carrying the selected target DNA nucleotide sequence.

23. A kit for rapidly detecting waterborne pathogens, such as bacteria, comprising:
a portable filtration device including a first port for receiving a liquid sample to be tested; a second port for venting air and a third port for discharging purified liquid, the portable filtration device including a plurality of hollow semi-permeable fibers for filtering the liquid sample and generating a filtrate within lumens of the hollow semi-permeable fibers, the filtrate containing the waterborne pathogens in a concentrated form;
a syringe configured for being sealingly mated to the first port and configured to deliver a lysing agent into lumens of the plurality of hollow semi-permeable fibers for lysing of the waterborne pathogens and formation of a lysed waterborne pathogen solution, the syringe further configured for removing the lysed waterborne pathogen solution from within the lumens; and
a molecular detection and amplification device for analyzing the lysed waterborne pathogen solution and detecting a presence of bacteria within the amplified lysed waterborne pathogen solution.

24. The kit of claim 23, wherein the amplified lysed waterborne pathogen solution comprises an amplified lysed bacterial solution.


SEQUENCE LISTING (3 sequences)

SEQ ID NO: 1 - DNA, 20 bp, Artificial Sequence
  Synthetic Primer
atgacgtcaagtcatcatgg

SEQ ID NO: 2 - DNA, 19 bp, Artificial Sequence
  Synthetic Primer
aggaggtgatccarccgca

SEQ ID NO: 3 - DNA, 22 bp, Artificial Sequence
  Synthetic Probe
caccgcccgtcacaccatggga